(19) United States
(12) Patent Application Publication
(10) Pub. No.: US 2011/0281740 A1
(43) Pub. Date: Nov. 17, 2011

(54) Methods for Real Time Single Molecule Sequencing

(76) Inventors: Joseph Beechem, Eugene, OR (US); Vi-En Choong, Carlsbad, CA (US); Theo Nikiforov, Carlsbad, CA (US)

(21) Appl. No.: 13/001,545

(22) PCT Filed: Jun. 30, 2009
(86) PCT No.: PCT/US09/49324

§ 371 Date: May 16, 2011

Related U.S. Application Data
(60) Provisional application No. 61/077,090, filed on Jun. 30, 2008.
(60) Provisional application No. 61/089,497, filed on Aug. 15, 2008.
(60) Provisional application No. 61/090,346, filed on Aug. 20, 2008.

(51) Int. Cl. C40B 30/00; C12Q 1/68

(52) U.S. Cl. 506/7; 435/6.1

(57) ABSTRACT

Provided herein are methods and compositions for real time single molecule sequencing of a polymeric molecule, such as a polynucleotide, by isolating the polymeric molecule in a nanofluidic device, subjecting it in situ to a polymerase reaction wherein various components of the polymerase reaction mixture are labeled, and determining the time-sequence of incorporation of monomeric subunits during the polymerization process.

This application claims provisional priority to U.S. provisional applications no. 61/077,090, filed Jun. 30, 2008; 61/089,497, filed Aug. 15, 2008; and 61/090,346, filed Aug. 20, 2008; all of which are incorporated by reference herein in their entirety.

### FIELD OF THE INVENTION

The present disclosure relates generally to real time single molecule sequencing. More particularly, the present disclosure relates to real time sequencing of a single nucleic acid molecule within a nanofluidic device.

### BACKGROUND OF THE INVENTION

One of the most widely studied biological polymers is deoxyribonucleic acid (DNA), and most DNA studies involve sequence analysis. Traditional sequencing methods, commonly referred to as “first-generation” methods, require large quantities of the target DNA molecule to be sequenced using time and resource intensive processes. For example, Maxam-Gilbert sequencing involves the chemical cleavage of end-labeled fragments of DNA. The resulting fragments are then size separated by gel electrophoresis, and the sequence of the original end-labeled fragments is determined by analyzing the pattern of fragments produced by the gel. Read lengths using this approach are typically limited to approximately 500 nucleotides. Furthermore, such methods are lengthy, and frequently require amplification of the target DNA to obtain sufficient amounts of starting material.

Other traditional DNA sequencing methodologies generally involve monitoring the activity of a sequencing enzyme, such as DNA polymerase, as it replicates a test DNA molecule by polymerizing monomeric subunits, such as dNTPs, to extend a primer into a newly synthesized DNA strand that complements the test molecule of interest. The polymerization products are analyzed after the sequencing reaction has been terminated, thereby adding to the length of the process. For example, Sanger-dideoxy sequencing involves elongation of an end-labeled nucleotide primer with random incorporation of chain terminating dideoxy nucleotides in four separate DNA polymerase reactions. As with the chemically cleaved DNA fragments in the Maxam-Gilbert method, the extension products must be size separated by gel electrophoresis and the nucleotide sequence may be determined from analyzing the pattern of fragments in the gel. Originally performed with radionucleotide labeled primers, today the use of four different fluorescently labeled dideoxynucleotides enables the sequencing reactions to be size separated in a single gel lane, facilitating automated sequence determination. Read lengths utilizing this approach are limited to approximately 1000 nucleotides, and the process can take a few hours to half a day to perform.

Collectively, these first-generation methods are hampered by the requirement for a relatively large amount of DNA substrate, the need for complex liquid handling steps, short read-lengths (typically on the order of 500-1000 nucleotides), and the complexity of the underlying biochemistry. In addition, these approaches are not well-suited for rapid sequencing of nucleic acid molecules. Thus, there is a need in the art for rapid polymeric sequencing methods and compositions, for example, sequencing from small amounts of target molecules or from a single nucleic acid molecule more rapidly than is currently feasible with conventional sequencing methods.

The last decade has seen the emergence of the so-called “next-generation” or “second generation” methods, characterized by increased sequencing throughput and data generation rates, associated with lower sequencing costs per base, faster throughput and greater sensitivity. Still, the goal of real-time sequencing of a single target molecule remains elusive.

More recently, so-called “third generation” sequencing methods seek to sequence single target molecules in real time. These methods involve the monitoring of signals emitted by luminophores, fluorophores or other labels attached to various components of the sequencing machinery during the sequencing reaction. Typically, these methods immobilize at least one component of the sequencing reaction such as the target nucleic acid or the polymerase, usually through attachment of the polymerase and/or template DNA to a solid support. In one example, the methods require confinement of the sequencing reaction and/or the zone of signal detection to a narrow fixed region, so as to minimize interference from the environment.

Accordingly, there remains a need in the art for methods and compositions for sequencing of single nucleic acid molecules in real time using long read lengths at high speed with low error rates while requiring little to no manipulation of the nucleic acid sample prior to analysis.

### SUMMARY OF THE INVENTION

Provided herein are methods and compositions that permit real-time or near real-time sequencing of nucleic acids. In particular, detection, such as optical detection, that discerns nucleotide identity as it is incorporated permits rapid, accurate, and long reads of nucleic acid templates. For example, the disclosed methods permit the sequencing of a whole chromosome using longer read lengths at higher speeds, thereby facilitating macroscale analysis of nucleic acid sequences for rapid and accurate identification of features as large repeats, inversions, indels and methylation patterns. Moreover, these methods readily facilitate high throughput sequencing in parallel, and ultimately allow the simultaneous sequencing of an entire genome rapidly and cheaply.

In some embodiments, provided herein is a method for genotyping or sequencing a target nucleic acid molecule, said method comprising: (a) immobilizing onto a solid support a target nucleic acid molecule, a polymerase, or a donor fluorophore; (b) subjecting the solid support to a polymerization reaction by contacting it with a mixture comprising sufficient components to permit nucleotide incorporation in the employed format including but not limited to a polymerase and at least one detectably-labeled nucleotide polyphosphate; (c) detecting a time sequence of incorporations of detectably-labeled nucleotide polyphosphates into a nascent nucleic acid molecule by detecting one or more detectable signals emitted during incorporation of one or more nucleotide polyphosphates; and (d) genotyping or sequencing said single target nucleic acid by converting the time sequence of detected signals into a sequence of the target nucleic acid molecule.

In some embodiments, provided herein is a method for genotyping or sequencing a nucleic acid molecule, said method comprising: (a) immobilizing onto a solid support a target nucleic acid molecule; (b) contacting said solid support with a polymerase and at least one detectably-labeled nucleotide polyphosphate under conditions where the at least one detectably-labeled nucleotide polyphosphate is incorporated into a growing nucleic acid molecule by the polymerase; (c) detecting a time sequence of incorporations of the at least one detectably-labeled nucleotide polyphosphate into the growing nucleic acid molecule; and (d) genotyping or sequencing said target nucleic acid by converting the detected time sequence of incorporations into a nucleic acid sequence.

In some embodiments, provided herein is a method for genotyping or sequencing a single target nucleic acid molecule, said method comprising: (a) immobilizing onto a solid support a target nucleic acid molecule; (b) contacting said solid support with a polymerase and at least one fluorescent terminally-labeled nucleotide polyphosphate; (c) optically detecting a time sequence of incorporation of the fluorescent terminally-labeled nucleotide polyphosphate into a growing nucleotide strand by detecting a time sequence of fluorescent signals emitted by the at least one fluorescent terminally-labeled nucleotide polyphosphate; and (d) genotyping or sequencing said single target nucleic acid by converting the time sequence of detected fluorescent signals into a nucleic acid sequence.

In some embodiments, provided herein is a method for determining in real time a nucleotide sequence of a nucleic acid molecule, comprising the steps of: (a) isolating a single nucleic acid molecule in a nanochannel of a nanofluidic device; (b) conducting in the nanochannel a polymerase reaction in the presence of at least one detectably-labeled nucleotide or nucleotide analog, which reaction results in the production of a detectable signal indicating incorporation of the at least one detectably-labeled nucleotide or nucleotide analog into a growing nucleotide strand by the polymerase; (c) detecting a time sequence of nucleotide or nucleotide analog incorporations; and (d) determining the identity of one or more nucleotides or nucleotide analogs incorporated during the polymerase reaction, thereby determining some or all of the nucleotide sequence of the nucleic acid molecule.

Optionally, the target nucleic acid, polymerase, donor fluorophore and/or any other suitable component of the polymerization machinery can be immobilized onto a solid support. In some embodiments, multiple target nucleic acid sequences are immobilized on a solid support to form a solid support comprising more than one site or location each comprising only one single individual molecule of target nucleic acid sequence. In other embodiments, the polymerase is attached to the solid support. In some embodiments, a donor fluorophore is attached to the solid support. In a specific embodiment, the donor fluorophore is operably linked to the polymerase.

In some embodiments, the detectable label comprises a fluorescent moiety. In some embodiments, the signal is a signal resulting from a nonradiative transfer of energy from a donor fluorophore to an acceptor fluorophore during the incorporation reaction, such as a FRET signal. The donor fluorophore can be operably linked on the polymerase or on the nucleic acid. In some embodiments, the donor fluorophore is a nanoparticle, a nanocrystal or a quantum dot.

In some embodiments, the detectable label is cleaved from the nucleotide upon incorporation into the growing strand. In a typical embodiment, the detectably labeled nucleotide polyphosphate can comprise a terminally-labeled nucleotide polyphosphate, i.e., a nucleotide polyphosphate comprising a detectable label operably linked to a terminal phosphate of the nucleotide. The term “terminal phosphate” and its variants, as used herein, refer to any phosphate within the nucleotide polyphosphate chain other than the alpha or beta phosphate. In some embodiments, the detectable label is attached to the γ-phosphate of the nucleotide polyphosphate, or to any other terminal phosphate of the nucleotide polyphosphate. In some embodiments terminally-labeled nucleotide polyphosphate has three or more phosphates. In other embodiments, the terminally-labeled nucleotide polyphosphate has four or more phosphates. In some embodiments, the nucleotide polyphosphate is not terminally-labeled, but rather labeled on an internal phosphate, for example, the α-phosphate, the β-phosphate, or another internal phosphate.

In one aspect, the polymerase, the nucleic acid molecule or the donor fluorophore is attached to a solid support. Any desired number of target molecules can be sequenced simultaneously while attached to the solid support. In some embodiments, the location of the individual molecules is addressable in the support. Any suitable solid support can be employed. In some embodiments, the solid support is glass, plastic, glass with surface modifications, silicon, metals, semiconductors, high refractive index dielectrics, nylon, nitrocellulose, PVDF, crystals, gels, and polymers. The solid support can be in any format including but not limited to a plate, microarray, sheet, filter, or beads.

In some embodiments, the detectably-labeled nucleotide comprises a nucleotide polyphosphate comprising a detectable label operably linked to the terminal phosphate of the nucleotide polyphosphate. Optionally, the detectable label can be a detectable label linked to a terminal phosphate in the polyphosphate chain of the detectably-labeled nucleotide, which reaction results in the production of a labeled polyphosphate that is released from the detectable terminal-phosphate labeled nucleotide.

In some embodiments, the nanofluidic device further comprises a nanochannel array. Typically, the nanochannel array can comprise 100, 1,000, 10,000, 100,000 or more nanochannels.

Optionally, the nanofluidic device can have one or more nanochannels having a trench width equal to or less than about 150, 100, 50 or 5 nanometers and/or a trench depth equal to or less than about 250, 50, 10 or 5 nanometers.

In some embodiments, the nanofluidic device can have one or more nanochannels capable of transporting a macromolecule across their length. Typically, the macromolecule is transported across the one or more nanochannels in an elongated form.

Optionally, the nanofluidic device may comprise one or more nanochannels formed by nanoimprint lithography, spin coating, electron beam lithography, focused ion beam milling, photolithography, reactive ion etching, wet etching, plasma-enhanced chemical vapor deposition, electron beam evaporation, sputter deposition, and combinations thereof.

Optionally, the nanofluidic device may comprise a nanofluidic area and a microfludic area. In some embodiments, the nanofluidic device may comprise a nanofluidic area and a microfludic area separated by a gradient interface. See, for example, U.S. Pat. No. 7,217,562.

Optionally, the detectable label of the detectably-labeled nucleotide can be a Forster resonance energy transfer (FRET) acceptor. In some embodiments, the detectable signal is produced as a result of Forster resonance energy transfer (FRET) from a FRET donor to the FRET acceptor.

Optionally, the detectable label attached to the nucleotide can be any suitable label that confers sufficient detection sensitivity within the assay format, including but not limited to a chromophore, fluorophore or luminophore. In some embodiments, the detectable label of the detectably-labeled nucleotide can be a fluorophore selected from the group consisting of: xanthine dye, fluorescein, cyanine, rhodamine, coumarin, acridine, Texas Red dye, BODIPY, ALEXA, GFP, and a derivative or modification of any of the foregoing.

Optionally, the nucleic acid polymerase of the nucleic acid polymerase reaction can be an RNA polymerase, DNA polymerase or reverse transcriptase. In some embodiments, the DNA polymerase of the nucleic acid polymerase reaction is a Klenow fragment of DNA polymerase I, E. coli DNA polymerase I, T7 DNA polymerase, T4 DNA polymerase, Thermus acquaticus DNA polymerase, or Thermococcus litoralis DNA polymerase.

Optionally, the nucleic acid polymerase of the nucleic acid polymerase reaction can be operably linked to a Forster resonance energy transfer (FRET) moiety. In some embodiments, the FRET moiety is a FRET donor. Optionally, the FRET donor can be a nanoparticle, nanocrystal or quantum dot.

In some embodiments, the FRET donor is a nanocrystal. Optionally, the nanocrystal can be surrounded with a coating material. In some embodiments, the coating material may comprise imidazole, histidine or carnosine.

Optionally, the nanocrystal may comprise a core comprising a first semiconductor material and a capping later deposited on the core comprising a second semiconductor material.

In some embodiments, the nanocrystal emits light with a quantum yield of greater than about 10%, 50%, or 70%.

In some embodiments, the nanocrystal further comprises cadmium selenide (CdSe), cadmium sulfide (CdS), cadmium telluride (CdTe), or mixtures thereof.

Optionally, the nanocrystal is a doped metal oxide nanocrystal.

In some embodiments, the nucleic acid polymerase of the nucleic acid polymerase reaction is further contacted with a nucleotide primer.

In some embodiments, the nucleotide primer is extended by a plurality of nucleotides. Typically, the nucleotide primer is extended by at least 100, 250, 500 or 1000 nucleotides.

In some embodiments, the nucleotide primer comprises at least 10, 25 or 50 nucleotides.

In some embodiments, the detectably labeled nucleotide has three, four or more phosphates.

In some embodiments, the rate of nucleotide sequence determination of a single nucleic acid molecule is equal to or greater than 0.1, 1, 10, 100 or 1000 bases per second.

In some embodiments, the error rate of nucleotide sequence determination is equal to or less than 25%, 10%, 5%, 3%, 1%, 0.1%, 0.01% and 0.001%.

Optionally, the nucleic acid molecule comprises chromosomal DNA. In some embodiments, the nucleic acid molecule comprises a complete and intact chromosome.

Also provided for herein is a method for determining the sequence of one or more additional nucleic acid molecules in parallel with determining the sequence of a first DNA molecule according to the methods provided herein.

### DESCRIPTION OF THE FIGURES

FIG. 1 shows a schematic of the single molecule sequencing reaction using a nanocrystal as the donor fluorophore using a nucleic acid attached to a solid substrate (A) or the donor fluorophore attached to a solid substrate (B).

FIG. 2 shows an exemplary correlation analysis.

### DETAILED DESCRIPTION OF THE INVENTION

Unless defined otherwise, all technical and scientific terms used herein have the same meaning as is commonly understood by one of ordinary skill in the art to which this disclosure belongs.

All patents, patent applications, published applications, treatises and other publications referred to herein, both supra and infra, are incorporated by reference in their entirety. If a definition and/or description is set forth herein that is contrary to or otherwise inconsistent with any definition set forth in the patents, patent applications, published applications, and other publications that are herein incorporated by reference, the definition and/or description set forth herein prevails over the definition that is incorporated by reference.

The practice of the present disclosure will employ, unless otherwise indicated, conventional techniques of molecular biology, microbiology and recombinant DNA techniques, which are within the skill of the art. Such techniques are explained fully in the literature. See, for example, Sambrook, J., and Russell, D. W., 2001, Molecular Cloning: A Laboratory Manual, Third Edition; Ausubel, F. M., et al., eds., 2002, Short Protocols In Molecular Biology, Fifth Edition.

As used herein, the term “a” or “an” means “at least one” or “one or more”.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, unrecited additives, components, integers, elements or method steps.

As used herein, the terms “a,” “an,” and “the” and similar referents used herein are to be construed to cover both the singular and the plural unless their usage in context indicates otherwise. Accordingly, the use of the word “a” or “an” when used in conjunction with the term “comprising” in the claims or specification may mean “one,” but it is also consistent with the meaning of “one or more,” “at least one,” and “one or more than one.”

As used herein, the term “operably link” refers to chemical fusion or bond or an association of sufficient stability to withstand conditions encountered in the method of nucleotide sequencing utilized, between a combination of different molecules such as, but not limited to: between a linker and a functionalized nanocrystal; between a linker and a nucleotide; and the like. For example, a functionalized nanocrystal-labeled primer template, is performed in such a way that the resultant labeled primer can readily serve to initiate a polymerization reaction by a polymerase. Reactive functionalities comprise bifunctional reagents/linker molecules, free chemical groups (e.g., thiol, or carboxyl, hydroxyl, amino, amine, sulfo, etc.), reactive chemical groups (reactive with free chemical groups), and a combination thereof. Exemplary embodiments include but are not limited to those described in U.S. Pat. No. 6,326,144.

The term “linker” refers to a compound or moiety that acts as a molecular bridge to operably link two different moieties or molecules. The two different moieties or molecules may be linked to the linker in a step-wise manner. There is no particular size or content limitations for the linker so long as it can fulfill its purpose as a molecular bridge suitable for use in primer extension, genotyping, sequencing or strand synthesis. Linkers are known to those skilled in the art to include, but are not limited to, chemical chains, chemical compounds (e.g., reagents), and the like. The linkers may include, but are not limited to, homobifunctional linkers and heterobifunctional linkers. Heterobifunctional linkers, well known to those skilled in the art, contain one end having a first reactive functionality to specifically link a first molecule, and an opposite end having a second reactive functionality to specifically link to a second molecule.

In some embodiments, the reactive functionalities of the linker can be selected from the group consisting of amino-reactive groups and thiol-reactive groups. That is, the linker should be able to function to operably link by interacting with either a free thiol group or a free amino group present on either or both of the functionalized nanocrystal and the nucleotide to be linked. Depending on such factors as the molecules to be linked, and the conditions in which the method of strand synthesis is performed, the linker may vary in length and composition for optimizing such properties as stability, resistance to certain chemical and/or temperature parameters, and of sufficient stereo-selectivity or size to operably link the label to the nucleotide such that the resultant labeled nucleotide may serve as a template for the initiation of a polymerization reaction. Such linkers can be employed using standard chemical techniques. Such linkers are known to those skilled in the art to include, but are not limited to, amine linkers for attaching labels to nucleotide (see, e.g., U.S. Pat. No. 5,151,507); a linker preferably contain a primary or secondary amine for operably linking a label to a nucleotide; and a rigid hydrocarbon arm added to a nucleotide base (see, e.g., Science 282:1020-21, 1998).

The term “nanoparticle” and its variants, as used herein, refer to any particle with at least one major dimension in the nanosize range. Typically, a nanoparticle has at least one major dimension ranging from about 1 to 1000 nm. Examples of nanoparticles include a nanocrystal, such as a core/shell nanocrystal, plus any tightly-associated organic coating or other material that may be on the surface of the nanocrystal. A nanoparticle may also include a bare core/shell nanocrystal, as well as a core nanocrystal or a core/shell nanocrystal having a layer of, e.g., TOPO or other material that is not removed from the surface by ordinary solvation. A nanoparticle may have a layer of ligands on its surface which may further be cross-linked; and a nanoparticle may have other or additional surface coatings that modify the properties of the particle, for example, solubility in water or other solvents. Such layers on the surface are included in the term ‘nanoparticle.’

The term “nanocrystal” and its variants, as used herein, refer to any nanoparticle made out of an inorganic substance that typically has an ordered crystalline structure. They can refer to a nanocrystal having a crystalline core, or to a core/shell nanocrystal, and may be 1-100 nm in its largest dimension, preferably about 1 to 50 nm in its largest dimension. A core nanocrystal is a nanocrystal to which no shell has been applied; typically it is a nanocrystal, and typically it is made of a single semiconductor material. It may be homogeneous, or its composition may vary with depth inside the nanocrystal.

The term “quantum dot” and its variants, as used herein, refer to any nanocrystalline particle made from a material that in the bulk is a semiconductor or insulating material, which has a tunable photophysical property in the near ultraviolet (UV) to far infrared (IR) range. Commercially available quantum dots include the QDot® nanocrystals supplied by Life Technologies Corp. (formerly known as Invitrogen Corp.)

As used herein, the term “incorporation” and its variants when used in reference to nucleotides or nucleotide analogs embraces any and all steps involved in nucleotide polymerization. Nucleotide polymerization is typically a multi-step process that includes binding of the polymerase to a template nucleic acid molecule, approach of a candidate nucleotide to be incorporated near to the polymerase active site, binding of the candidate nucleotide within the polymerase active site, interrogation of the candidate nucleotide for complementarity with the template nucleotide on the target or template nucleic acid molecule, catalysis of a nucleotidyl transferase reaction involving phosphodiester bond formation between the terminal end of the extending nucleic acid strand and the candidate nucleotide, and cleavage and liberation of a polyphosphate chain derived from the incorporated nucleotide, which typically diffuses away from the polymerase. The entire process typically repeats, resulting in successive incorporations of multiple nucleotides onto the end of the extending nucleic acid molecule. Alternatively, in some instances, the nucleotide may dissociate from the polymerase active site and diffuse away unchanged, without occurrence of the nucleotidyl transferase reaction (a so-called “non-productive binding” event). As used herein, the term “nucleotide incorporation” and its variants comprises both productive and non-productive binding events, including but not limited to events starting from approach and binding of the candidate nucleotide with the polymerase and all subsequent events through and including phosphodiester bond formation, cleavage and liberation of the polyphosphate chain, or alternatively dissociation of the intact and unchanged nucleotide from the polymerase active site, and diffusion of the released polyphosphate (or the intact and unchanged nucleotide) away from the polymerase.

Disclosed herein are sequencing methods and compositions that collectively provide rapid sequencing of a single polymeric molecule of interest, such as a nucleic acid, by monitoring of signals emitted.

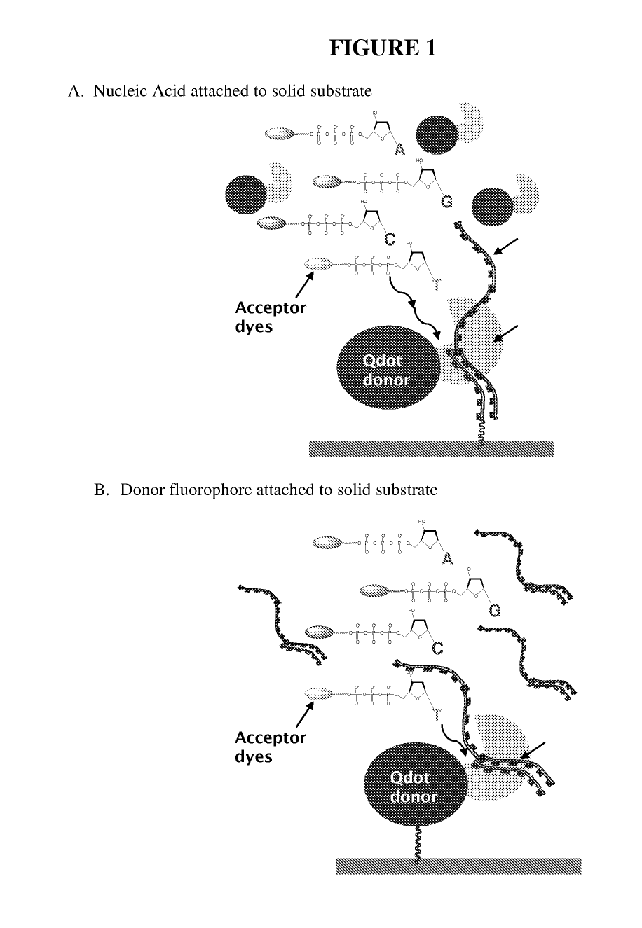

In some embodiments, provided herein is a method for genotyping or sequencing a single target nucleic acid molecule, said method comprising: (a) immobilizing onto a solid support a target nucleic acid molecule, a polymerase, or a donor fluorophore; (b) contacting said solid support with a polymerization reaction mixture comprising sufficient components to permit incorporation events in the employed format including but not limited to a polymerase and at least one fluorescent terminally-labeled nucleotide polyphosphate; (c) optically detecting a time sequence of incorporation of the fluorescent terminally-labeled nucleotide polyphosphates into the growing nucleotide strand, by detecting a change or presence of fluorescent signals emitted by the fluorescent label of the at least one fluorescent terminally-labeled nucleotide; and (d) genotyping or sequencing said single target nucleic acid by converting the sequence of the fluorescent signals detected during the polymerization reaction into a nucleic acid sequence. In some embodiments, the target nucleic acid, polymerase or donor fluorophore can be immobilized onto a solid support. In some embodiments, more than one target nucleic acid sequence are operably linked to the solid support so as to form a solid support comprising more than one site or location, each such site or location comprising only one single individual sequencing site. See, for example, FIG. 1.

In some embodiments, provided herein is method for genotyping or sequencing a nucleic acid molecule, said method comprising: (a) immobilizing onto a solid support a target nucleic acid molecule; (b) contacting said solid support with a polymerase and at least one detectably-labeled nucleotide polyphosphate under conditions where the at least one detectably-labeled nucleotide polyphosphate is incorporated into a growing nucleic acid molecule by the polymerase; (c) detecting a time sequence of incorporations of the at least one detectably-labeled nucleotide polyphosphate into the growing nucleic acid molecule; and (d) genotyping or sequencing said target nucleic acid by converting the detected time sequence of incorporations into a nucleic acid sequence.

In some embodiments, provided herein is a method for genotyping or sequencing a single target nucleic acid molecule, said method comprising: (a) immobilizing onto a solid support a target nucleic acid molecule; (b) contacting said solid support with a polymerase and at least one fluorescent terminally-labeled nucleotide polyphosphate; (c) optically detecting a time sequence of incorporation of the fluorescent terminally-labeled nucleotide polyphosphates into a growing nucleotide strand by detecting a time sequence of fluorescent signals emitted by the at least one fluorescent terminally-labeled nucleotide polyphosphate; and (d) genotyping or sequencing said single target nucleic acid by converting the time sequence of detected fluorescent signals into a nucleic acid sequence.

In some embodiments, the methods disclosed herein involve the isolation and in situ sequencing of a single polymeric molecule of interest within a nanofluidic device. During the polymerization process, the labels on various reaction components emit detectable signals, which can be detected and analyzed to determine the time-sequence of incorporation events.

In some embodiments, the methods used to detect and monitor progress of the sequencing reaction are based on detection of signals resulting from Forster Resonance Energy Transfer (FRET). As discussed below, fluorescence resonance energy transfer (FRET) is a distance-dependent interaction between the electronic excited states of two molecules, during which energy is transferred non-radiatively from the first excited molecule (called a FRET donor) to the second molecule, called a FRET acceptor, which may then emit a photon. The process of energy transfer results in a reduction (quenching) of fluorescence intensity and excited state lifetime of the FRET donor, and can produce an increase in the emission intensity of the FRET acceptor. FRET occurs only when two appropriately labeled molecules or moieties are sufficiently proximal to each other to transfer energy.

In one exemplary embodiment, the polymeric molecule of interest (which can be referred to as the “template”) is contacted with a polymerase reaction comprising a polymerase and individual monomers capable of polymerization by the polymerase. The polymerase is operably linked or otherwise labeled with a moiety capable of acting as a FRET donor, for example a detectable nanoparticle, and the monomers are each labeled with different moieties capable of acting as FRET acceptors. In some embodiments, a polymerase molecule typically attaches to priming sites within the polymeric template, and then binds to an incoming labeled monomer in a template-dependent fashion. When the polymerase binds to the incoming labeled monomer, the nanoparticle attached to the polymerase is brought into proximity with the FRET acceptor of the monomer and FRET occurs, resulting in localized and detectable FRET emission events that permit monitoring of each localized sequencing reaction in situ. As the polymerase extends the newly synthesized strand by adding labeled monomers to the free 3′ end of the strand in a template-dependent fashion, the identity of each successive incoming monomer bound and incorporated by the polymerase will be identifiable by the emission spectrum of the FRET acceptor attached to that particular monomer. Accordingly, the monomer can be identified by optical or other suitable detection and characterization of the FRET signal, as described below.

In some embodiments, the polymerase can be labeled with a nanoparticle, typically a nanocrystal and even more typically a quantum dot. In some embodiments, the nanoparticle, nanocrystal or quantum dot is fluorescent.

Typically, the polymer to be sequenced is a nucleic acid, the polymerase is a nucleotide polymerase such as DNA polymerase, RNA polymerase or reverse transcriptase, and the monomers are nucleotides or nucleotide analogs.

Typically, the polymeric molecule to be sequenced is a nucleic acid. Suitable nucleic acid molecules that can be sequenced according to the present disclosure include without limitation single-stranded DNA, double-stranded DNA, single stranded DNA hairpins, DNA/RNA hybrids, RNA with an appropriate polymerase recognition site, and RNA hairpins. In one preferred embodiment, the polymer is DNA, the polymerase is a DNA polymerase or an RNA polymerase, and the labeled monomer is a nucleotide, a nucleotide polyphosphate, or an analog. In another preferred embodiment, the polymer to be sequenced is RNA and the polymerase is reverse transcriptase.

Any suitable polymerase may be used that is capable of polymerizing monomeric subunits into polymers. Preferably, the polymerase is a nucleotide polymerase, i.e., a polymerase that can polymerize nucleotides. Generally, the nucleotide polymerase will elongate a pre-existing polynucleotide strand, typically a primer, by polymerizing nucleotides on to the 3′ end of the strand. Exemplary polymerases include without limitation DNA polymerases, RNA polymerases and reverse transcriptases. In a preferred embodiment, the polymerase is a DNA polymerase. Suitable nucleotide polymerases that may be used to practice the methods disclosed herein include without limitation any naturally occurring nucleotide polymerases as well as mutated, truncated, modified, genetically engineered or fusion variants of such polymerases. Known conventional naturally occurring DNA polymerases include without limitation bacterial DNA polymerases, eukaryotic DNA polymerases, archaeal DNA polymerases, viral DNA polymerases and phage DNA polymerases. Suitable bacterial DNA polymerase include without limitation E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Suitable eukaryotic DNA polymerases include without limitation the DNA polymerases α, δ, ∈, η, ζ, β, σ, λ, μ, ι, and κ, as well as the Rev1 polymerase (terminal deoxycytidyl transferase) and terminal deoxynucleotidyl transferase (TdT). Suitable viral DNA polymerases include without limitation T4 DNA polymerase, Phi29 DNA polymerase and T7 DNA polymerase. Suitable archaeal DNA polymerases include without limitation the thermostable and/or thermophilic DNA polymerases such as, for example, DNA polymerases isolated from Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavus (Tfl) DNA polymerase, Pyrococcus woesei (Pwo) DNA polymerase, Pyrococcus furiosus (Pfu) DNA polymerase as well as Turbo Pfu DNA polymerase, Thermococcus litoralis (Tli) DNA polymerase or Vent DNA polymerase, Pyrococcus sp. GB-D polymerase, “Deep Vent” DNA polymerase, New England Biolabs), Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. 9° N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pernix DNA polymerase; and the heterodimeric DNA polymerase DP1/DP2.

In some embodiments, the polymerase can be, for example, a polymerase isolated from a phototrophic and/or halotrophic organism. The polymerase can be a polymerase isolated from Cyanophage S-CBP1, Cyanophage S-CBP2, Cyanophage S-CBP3, Cyanophage Syn5, Cyanophage S-CBP42, Synechococcus phage P60, Roseobacter phage S100 DNA Polymerase, Oedogonium cardiacum chloroplast DNA Polymerase, Salterprovirus His1 Polymerase, Salterprovirus His2 Polymerase, Ostreococcus tauri V5, Ectocarpus siliculosus virus 1 or any combination of such polymerases.

Similarly, suitable RNA polymerases include, without limitation, T7, T3 and SP6 RNA polymerases. Suitable reverse transcriptases include without limitation reverse transcriptases from HIV, HTLV-I, HTLV-II, FeLV, FIV, SIV, AMV, MMTV and MoMuLV, as well as the commercially available “Superscript” reverse transcriptases, (Invitrogen) and telomerases. In addition to naturally occurring polymerases, the methods and systems disclosed herein may also be practiced using any subunits, mutated, modified, truncated, genetically engineered or fusion variants of naturally occurring polymerases (wherein the mutation involves the replacement of one or more or many amino acids with other amino acids, the insertion or deletion of one or more or many amino acids, or the conjugation of parts of one or more polymerases) non-naturally occurring polymerases, synthetic molecules or any molecular assembly that can polymerize a polymer having a pre-determined or specified or templated sequence of monomers may be used in the methods disclosed herein.

In particular, polymerases that retain the desired levels of processivity when conjugated to a donor or acceptor fluorophore are preferred. Also preferred are polymerases that are selected and/or engineered to exhibit high fidelity with low error rates. The term “fidelity” as used herein refers to the accuracy of nucleotide polymerization by a given template-dependent nucleotide polymerase. The fidelity of a nucleotide polymerase is typically measured as the error rate, i.e., the frequency of incorporation of a nucleotide in a manner that violates the widely known Watson-Crick base pairing rules. The accuracy or fidelity of DNA polymerization is influenced not only by the polymerase activity of a given enzyme, but also by the 3′-5′ exonuclease activity of a DNA polymerase. The fidelity or error rate of a DNA polymerase may be measured using any suitable assay. See, for example, Lundburg et al., 1991 Gene, 108:1-6. By suitable selection and engineering of the nucleotide polymerase, the error rate of the single-molecule sequencing methods disclosed herein can be further reduced.

Any suitable nucleotides or nucleotide analogs may be used for the disclosed methods and compositions. The terms “nucleotide” or “nucleotide analogs” or their variants, as used herein, refer to any compounds that can be polymerized and/or incorporated into a newly synthesized strand by a naturally occurring, genetically modified or engineered nucleotide polymerase. Examples of nucleotide compounds that may be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, and modified phosphate-sugar backbone nucleotides, and any analogs or variants of the foregoing.

Any detectable label that is suitable for attachment to the polymerase and/or the nucleotides may be used, including but not limited to luminescent, photoluminescent, electroluminescent, bioluminescent, chemiluminescent, fluorescent and/or phosphorescent labels. Typically, the label comprises a FRET donor and/or a FRET acceptor. The FRET donor and/or the FRET acceptor is typically a fluorophore or fluorescent label; however the FRET donor and/or FRET acceptor may also be a luminophore, chemiluminophore, bioluminophore or other label, or a quencher that can participate in this reaction, as described below. In this description, the FRET labels may be referred to as fluorophores or fluorescent labels for convenience, but this in no way is meant to exclude the possibility of using a quencher or limit the donor and/or acceptor only to fluorescent labels. Alternatively, the detectable labels used in the disclosed methods and compositions may undergo other types of energy transfer with each other, including but not limited to luminescence resonance energy transfer, bioluminescence resonance energy transfer, chemiluminescence resonance energy transfer, and similar types of energy transfer not strictly following the Forster's theory, such as the nonoverlapping energy transfer when nonoverlapping acceptors are utilized. See, for example, Anal. Chem. 2005, 77: 1483-1487.

According to the present disclosure, the polymerase and the nucleotides can be operably linked to their corresponding labels using suitable methods. As used herein, the term “operably link” and its variants refer to chemical fusion or bonding or association of sufficient stability to withstand conditions encountered in the method of nucleotide sequencing utilized, between a combination of different molecules such as, but not limited to: between a linker and a functionalized nanocrystal; between a linker and a protein; and the like. For example, a functionalized nanocrystal-labeled polymerase is operably linked in such a way that the resultant labeled polymerase can readily participate in a polymerization reaction. See, for example, Hermanson, G., 2008, Bioconjugate Techniques, Second Edition. Suitable linkers include, for example, any compound or moiety that can act as a molecular bridge to operably link two different molecules. Exemplary linkers include, but are not limited to, chemical chains, chemical compounds (e.g., reagents), and the like. The linkers may include, but are not limited to, homobifunctional linkers and heterobifunctional linkers. For example, heterobifunctional linkers contain one end having a first reactive functionality to specifically link to a first molecule, and an opposite end having a second reactive functionality to specifically link to a second molecule. Depending on such factors as the molecules to be linked and the conditions in which the method of strand synthesis is performed, the linker may vary in length and composition for optimizing properties such as stability, length, FRET efficiency, resistance to certain chemicals and/or temperature parameters, and be of sufficient stereo-selectivity or size to operably link a nanoparticle, nanocrystal, quantum dot or other label to a polymerase or nucleotide such that the resultant conjugate is useful in optimizing a polymerization reaction. Linkers can be employed using standard chemical techniques and include but not limited to, amine linkers for attaching labels to nucleotides (see, for example, U.S. Pat. No. 5,151,507); a linker typically contain a primary or secondary amine for operably linking a label to a nucleotide; and a rigid hydrocarbon arm added to a nucleotide base (see, for example, Science 282:1020-21, 1998

In a preferred embodiment, the detectable label of the polymerase is a nanoparticle, a nanocrystal or a quantum dot. Any suitable nanoparticle, nanocrystal or quantum dot can be employed to label the polymerase, nucleotides or any other suitable component, for example the primer and/or nucleic acid template, of the sequencing machinery according to the present disclosure. Such nanoparticles can be made by any suitable methods. Optionally, the nanoparticle comprises a nanocrystal core and shell, which can be made of any suitable metal and non-metal atoms that are known to form semiconductor nanocrystals. Semiconductor nanocrystals may be made using any suitable technique including but not limited to those disclosed in Murray et al., 1993, J. Am. Chem. Soc. 115:8706-8715; Hines et al., 1996, J. Phys. Chem. 100:468-71; Peng et al., 1997, J. Am. Chem. Soc. 119:7019-29, U.S. Pat. Nos. 6,048,616, 5,990,479, 5,690,807, 5,505,928 and 5,262,357, as well as International Patent Publication No. WO 99/26299, published May 27, 1999. These methods typically produce nanocrystals having a coating of hydrophobic ligands on their surfaces which protect them from rapid degradation. Generally, fabrication methods produce two distinct layers, a core and a shell, in separate steps, but other methods can also be used.

In some embodiments, the nanoparticles are bright fluorescent nanoparticles, e.g., having a quantum yield of at least about 20%, sometimes at least 30%, sometimes at least 40%, and sometimes at least 50% or greater. In some embodiments, the nanoparticles comprise a surface layer of ligands to protect the nanocrystal from degradation in use or during storage.

Any suitable nanoparticle can be used as a label in the disclosed methods and compositions. In some embodiments, the nanoparticle can comprise a nanocrystal. Exemplary nanocrystals include without limitation those described in U.S. Pat. Nos. 5,505,928; 5,990,479; 6,114,038; 6,207,229; 6,207,392; 6,251,303; 6,319,426; 6,444,143; 6,274,323; 6,306,610; 6,322,901; 6,326,144; 6,423,551; 6,699,723; 6,426,513; 6,500,622; 6,548,168; 6,576,291; 6,649,138; 6,815,064; 6,819,692; 6,821,337; 6,921,496; 7,138,098; 7,068,898; 7,079,241; and 7,108,915.

It is also contemplated that particles with nanoparticle-like functions can serve as a donor fluorophore. For example, one may employ a bead filled with organic dyes as the donor fluorophore.

In particular, exemplary materials for use as nanoparticles in the biological and chemical assays disclosed herein include, but are not limited to, ones including Group 2-16, 12-16, 13-15 and 14 element-based semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, PbS, PbSe, Ge and Si and ternary and quaternary mixtures thereof. In some embodiments, the nanocrystal has a core of CdX wherein X is Se (Cadmium Selenide), Te (Cadmium Telluride) or S (Cadmium Sulfide). In other embodiments, the nanoparticle comprises a doped metal oxide nanocrystal.

The nanoparticles of the present disclosure can comprise a core/shell nanocrystal having a nanocrystal core covered by a semiconductor shell. The thickness of the shell can be adapted to provide desired particle properties. The thickness of the shell affects fluorescence wavelength slightly, and has substantial effects on the quantum yield, fluorescence stability, and other photostability characteristics. In some embodiments, the nanocrystal has a semiconductor shell up to about 5 monolayers in thickness, or up to about 3 nm in thickness. In some embodiments, shells ranging from 4-6 monolayers of CdS and 2.5-4.5 monolayers of ZnS may be used. In some embodiments, the shell is thinner, and can be up to about one monolayer in thickness, or up to about 2 monolayers in thickness.

In some embodiments, the nanoparticle comprises a core semiconductor nanocrystal that is modified to enhance the efficiency and stability of its fluorescence emissions, prior to ligand modifications described herein, by adding an overcoating layer or shell to the semiconductor nanocrystal core. Having a shell may be preferred, because surface defects at the surface of the semiconductor nanocrystal can result in traps for electrons, or holes that degrade the electrical and optical properties of the semiconductor nanocrystal core, or other non-radiative energy loss mechanisms that either dissipate the energy of an absorbed photon or at least affect the wavelength of the fluorescence emission slightly, resulting in broadening of the emission band. An insulating layer at the surface of the semiconductor nanocrystal core can provide an atomically abrupt jump in the chemical potential at the interface that eliminates energy states that can serve as traps for the electrons and holes. This results in higher efficiency in the luminescent processes.

Suitable materials for the shell include semiconductor materials having a higher bandgap energy than the semiconductor nanocrystal core. In addition to having a bandgap energy greater than the semiconductor nanocrystal core, suitable materials for the shell should have good conduction and valence band offset with respect to the core semiconductor nanocrystal. Thus, the conduction band is desirably higher and the valence band is desirably lower than those of the core semiconductor nanocrystal. For semiconductor nanocrystal cores that emit energy in the visible (e.g., CdS, CdSe, CdTe, ZnSe, ZnTe, GaP, GaAs) or near IR (e.g., InP, InAs, InSb, PbS, PbSe), a material that has a bandgap energy in the ultraviolet regions may be used. Exemplary materials include ZnS, GaN, and magnesium chalcogenides, e.g., MgS, MgSe, and MgTe. For a semiconductor nanocrystal core that emits in the near IR, materials having a bandgap energy in the visible, such as CdS or CdSe, may also be used. The preparation of a coated semiconductor nanocrystal may be found in, e.g., Dabbousi et al. (1997) J. Phys. Chem. B 101:9463, Hines et al. (1996) J. Phys. Chem. 100: 468-471, Peng et al. (1997) J. Am. Chem. Soc. 119:7019-7029, and Kuno et al. (1997) J. Phys. Chem. 106:9869. It is also understood in the art that the actual fluorescence wavelength for a particular nanocrystal core depends upon the size of the core as well as its composition, so the categorizations above are approximations, and nanocrystal cores described as emitting in the visible or the near IR can actually emit at longer or shorter wavelengths depending upon the size of the core.

In some embodiments, the nanoparticle comprises a nanocrystal having metal atoms of a shell layer that are selected from Cd, Zn, Ga and Mg. The second element in these semiconductor shell layers can be selected from S, Se, Te, P, As, N and Sb. In some embodiments, the semiconductor nanocrystal is a core/shell nanocrystal, and the core comprises metal atoms selected from Zn, Cd, In, Ga, and Pb. Some preferred nanocrystal cores include CdS, CdSe, InP, CdTe, ZnSe and ZnTe; and some preferred shell materials include ZnS, ZnSe, CdS, and CdSe.

Optionally, the nanoparticle comprises a nanocrystal that is surrounded with a coating material. The coating may be made of any suitable material, such as, for example, imidazole, histidine or carnosine. CdX nanocrystals can be passivated with an overlayering (“shell”) uniformly deposited thereon. An exemplary passivating shell can comprise YZ wherein Y is Cd or Zn, and Z is S, or Se. The nanocrystals useful in the claimed methods may be functionalized to be water-soluble nanocrystals. “Water-soluble” is used herein to mean that the nanocrystals are sufficiently soluble or suspendable in an aqueous-based solution including, but not limited to, water, water-based solutions, and buffer solutions, which are used in one or more processes such as sequence determination. In some embodiments, the CdX core/YZ shell nanocrystals are overcoated with trialkylphosphine oxide, with the alkyl groups most commonly used being butyl and octyl.

The nanoparticle can be of any suitable size; typically, it is sized to provide fluorescence in the UV-Visible portion of the electromagnetic spectrum, since this range is convenient for use in monitoring biological and biochemical events in relevant media. The relationship between size and fluorescence wavelength is well known, thus making nanoparticles smaller may require selecting a particular material that gives a suitable wavelength at a small size, such as InP as the core of a core/shell nanoparticle designed to be especially small. Typically the nanoparticles of interest are from about 1 nm to about 100 nm in diameter, or from about 1 to about 50 nm, or from about 1 to about 40 nm, or from about 1 to about 25 nm. For a nanoparticle that is not substantially spherical, e.g. rod-shaped, it may be from about 1 to about 100 nm, or from about 1 to about 50 nm, or from about 1 to about 40 nm, or from about 1 nm to about 20 nm in its largest dimension.

Where a nanoparticle comprising a core/shell fluorescent semiconductor nanocrystal is used, it is sometimes advantageous to make the nanoparticle as small as practical; thus in some embodiments, the nanoparticle is less than about 10 nm in diameter, and often less than about 8 nm, and sometimes less than about 6 nm in diameter, and in some embodiments, the nanoparticle is less than about 5 nm in diameter or size, or less than 4 nm in diameter or size.

In certain embodiments, the nanoparticle comprises a quantum dot (QDOT) available from commercial manufacturers such as QDOT® nanocrystals from Invitrogen Corp. (Carlsbad, Calif.). Quantum dots typically comprise a semiconductor nanocrystal with size-dependent optical and electronic properties. In particular, the band gap energy of a quantum dot varies with the diameter of the crystal. QDOT nanocrystals are typically nanometer-scale atom clusters comprising a core, shell, and coating. The core is typically made up of a few hundred to a few thousand atoms of a semiconductor material, for example, cadmium mixed with selenium or tellurium. A semiconductor shell, for example, zinc sulfide, can surround and stabilize the core, improving both the optical and physical properties of the material. Typically, an amphiphilic polymer coating then encases this core and shell, providing a water-soluble surface that may be differentially modified to create QDOT nanocrystals that meet specific assay requirements. The amphiphilic inner coating may be covalently modified with a functionalized polyethylene glycol (PEG) outer coating. The PEG surface may reduce nonspecific binding in flow cytometry and imaging assays, thereby improving signal-to-noise ratios and providing clearer resolution of cell populations and cellular morphology. QDOT primary and secondary antibody conjugates, QDOT streptavidin conjugates, QTRACKER non-targeted quantum dots, and QDOT ITK amino (PEG) quantum dots, as well as the reactive nanocrystals provided in the QDOT Antibody Conjugation Kit (Invitrogen), utilize this PEG chemistry.

Useful quantum dots include those which are functionalized (a) to be water-soluble, and (b) to further comprise a protein or peptide which is operably linked to the quantum dot. Desirable features of the basic quantum dots themselves include that the class of quantum dots can be excited with a single excitation light source resulting in a detectable fluorescence emission of high quantum yield (for example, a single quantum dot having a fluorescence intensity that may be a log or more greater than that of a molecule of a conventional fluorescent dye) and with a discrete fluorescence peak.

For use in the disclosed compositions and methods, the nanoparticle can have any suitable surface chemistry that permits the attachment of the nanoparticle or quantum dot to the biological molecule of interest. For example, the nanoparticle can be a quantum dot with a carboxyl-derivatized amphiphilic coating that can be coupled to amines, hydrazines, or hydroxylamines using an EDAC (1-ethyl-3-(3-dimethylaminopropyl) carbodiimide hydrochloride) mediated reaction. Amino-derivatized coatings permit crosslinking with amine reactive groups such as isothiocyanates, succinimidyl esters and other active esters. Finally, quantum dots coated with covalently bound streptavidin or PEG enable linking to biotinylated molecules. See, for example, U.S. Pat. Nos. 6,251,303; 6,274,323; and 6,306,610.

Quantum dots have been successfully used for FRET detection in biological systems. See, for example, Willard et al., 2001, Nano. Lett. 1:469; Patolsky, F., et al., 2003, J. Am. Chem. Soc. 125:13918; Medintz, I. L., et al., 2003, Nat. Mater. 2:630; Zhang, C. Y., et al., 2005, Nat. Matter. 4:826. Quantum dots make particularly good FRET donors for several reasons. For example, quantum dot emission may be size-tuned to improve spectral overlap with any particular acceptor chromophore or quencher, and quantum dots also have greater quantum yields and are less susceptible to photobleaching than traditional FRET donors. Together, these characteristics enable greater FRET efficiencies and make continuous monitoring (such as real time monitoring) for FRET interactions possible.

Because nanoparticles are typically larger than traditional organic fluorescent dyes, the size of the nanoparticle relative to the R₀ of the FRET donor-acceptor pair should also be taken into consideration. For nanoparticles size-tuned to emit in the visible light spectrum, the radius from the nanoparticle's energy-transferring core to its surface typically ranges from 2 to 5 nm. Given typical R₀ distances of 5-10 nm, this means that acceptor chromophores must be within a few nanometers of the nanoparticle surface for efficient FRET between common donor-acceptor pairs. Larger nanoparticles may have R₀ distances that will fall within the shell of the dot itself, precluding efficient FRET. These spatial constraints are especially important when the nanoparticle is used to monitor interaction between a protein, nucleic acid, or some other molecule conjugated to the nanoparticle surface and the acceptor molecule. Interaction between the conjugated molecule and the acceptor must position the FRET acceptor close enough to the nanoparticle to allow FRET that is sufficient for detection.

Typically, the polymerase is operably linked to a nanoparticle using linkers and/or spacers as described herein. Alternatively, the polymerase may be linked to the nanoparticle using affinity coupling without the need for spacers. See, for example, Goldman et al., 2005, Anal. Chim. Acta 534:63-67.

Nucleotides that may be used in the nucleic acid polymerase reaction may be any compounds that can be polymerized and/or incorporated into an elongating polynucleotide chain by a polymerase, including but not limited to ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, and modified phosphate-sugar backbone nucleotides, and any analogs or variants of the foregoing. For sequencing of non-nucleic acid polymers, for example, a protein, any suitable monomers capable of polymerization by a naturally occurring, genetically engineered, or synthetic polymerase may be used, including, for example, amino acids (natural or synthetic) for protein or protein analog synthesis, and mono saccharides or poly saccharides for carbohydrate synthesis. In some embodiments, the labeled nucleotide monomer has three, four or more phosphates.

Preferably, the nucleotide is conjugated or otherwise operably linked to a detectable label. For example, dye labels may be conjugated to the terminal phosphate of deoxyribonucleotide polyphosphates using a linker and/or spacer using suitable techniques. Any suitable methods for detectably labeling nucleotides may be employed including but not limited to those described in U.S. Pat. Nos. 7,041,892, 7,052,839, 7,125,671 and 7,223,541; U.S. Pub. Nos. 2007/072196 and 2008/0091005; Sood et al., 2005, J. Am. Chem. Soc. 127:2394-2395; Arzumanov et al., 1996, J. Biol. Chem. 271:24389-24394; and Kumar et al., 2005, Nucleosides, Nucleotides & Nucleic Acids, 24(5):401-408. Suitable labels that may be used in the disclosed methods and conjugated, associated or otherwise operable linked to the polymerase or the nucleotides include any molecule, nano-structure, or other chemical structure that capable of being detected by a detection system, including but not limited to fluorescent dyes.

Typically, the FRET acceptor label is attached to a nucleotide phosphate group that is cleaved and released upon incorporation of the underlying nucleotide into the primer strand, for example the β-phosphate, the γ-phosphate, or the terminal phosphate of the incoming nucleotide. By cleaving the phosphate and releasing the label upon incorporation of the incoming nucleotide, the signal from the label (or, for embodiments wherein the label is a FRET donor, the FRET signal between the FRET donor and the FRET acceptor moieties) ceases after the nucleotide is incorporated and the label (or FRET signal) diffuses away. Thus, in these embodiments, a detectable signal indicative of nucleotide incorporation is generated as each incoming nucleotide hybridizes to a complementary nucleotide in the target nucleic acid molecule and becomes incorporated into the newly synthesized strand. By releasing the label upon incorporation, successive extensions can each be detected without interference from nucleotides previously incorporated into the complementary strand. Alternatively, the nucleotide may be labeled with a FRET acceptor moiety on an internal phosphate, for example, the alpha phosphate, the beta phosphate, or another internal phosphate.

When conducting FRET-based sequencing according to the methods described herein, donor-acceptor pairs are typically selected such that there is overlap between the emission spectrum of the donor and excitation spectrum of the acceptor. Any suitable FRET donor:acceptor pair may be used in the disclosed methods and compositions, including but not limited to a fluorescein, cyanine, rhodamine, coumarin, acridine, Texas Red dye, BODIPY, Alexa Fluor, GFP, or a derivative or modification of any of the foregoing. See, for example, U.S. Pub. No. 2008/0091995.

Although the energy transfer from the donor to the acceptor does not involve emission of light, it may be thought of in the following terms: excitation of the donor produces energy in its emission spectrum that is then picked up by the acceptor in its excitation spectrum, leading to the emission of light from the acceptor in its emission spectrum. In effect, excitation of the donor sets off a directed migration of energy, leading to emission from the acceptor when the two are sufficiently close to each other.

In addition to spectral overlap between the donor and acceptor, other factors affecting FRET efficiency include the quantum yield of the donor and the extinction coefficient of the acceptor. The FRET signal may be maximized by selecting high yielding donors and high absorbing acceptors, with the greatest possible spectral overlap between the two. See, e.g., Piston, D. W., and Kremers, G. J., 2007, Trends Biochem. Sci., 32:407.

In other embodiments, the label operably linked or attached to the nucleotide may be a quencher. Quenchers are useful as acceptors in FRET applications, because they produce a signal through the reduction or quenching of fluorescence from the donor fluorophore. As with conventional fluorescent labels, quenchers have an absorption spectrum and large extinction coefficients, however the quantum yield for quenchers is extremely reduced, such that the quencher emits little to no light upon excitation. For example, in a FRET detection system, illumination of the donor fluorophore excites the donor, and if an appropriate acceptor is not close enough to the donor, the donor emits light. This light signal is reduced or abolished when FRET occurs between the donor and a quencher acceptor, resulting in little or no light emission from the quencher. Thus, interaction or proximity between a donor and quencher-acceptor may be detected by the reduction or absence of donor light emission. For an example of the use of a quencher as an acceptor with a nanoparticle donor in a FRET system, see Medintz, I L et al. (2003) Nat. Mater. 2:630, herein incorporated by reference in its entirety. Examples of quenchers include the QSY dyes available from Molecular Probes (Eugene, Oreg.).

One exemplary method involves the use of quenchers in conjunction with fluorescent labels. In this strategy, certain nucleotides in the reaction mixture are labeled with a fluorescent label, while the remaining nucleotides are labeled with one or more quenchers. Alternatively, each of the nucleotides in the reaction mixture is labeled with one or more quenchers. Discrimination of the nucleotide bases is based on the wavelength and/or intensity of light emitted from the FRET acceptor, as well as the intensity of light emitted from the FRET donor. If no signal is detected from the FRET acceptor, a corresponding reduction in light emission from the FRET donor indicates incorporation of a nucleotide labeled with a quencher. The degree of intensity reduction may be used to distinguish between different quenchers.

Another exemplary method involves modulating FRET efficiency by varying the distance between the nanoparticle donor and the fluorescent label or quencher acceptor. In this strategy, the same type of fluorescent label or quencher may be used, however, the distance between the nanoparticle and the label is varied for each nucleotide to be identified, causing a modulation of FRET efficiency. The distance may be varied through the structure of the nucleotide itself, the position of the fluorescent label or quencher on the nucleotide, or the use of spacers or linkers during attachment of the fluorescent label or quencher to the nucleotide. Modulation of FRET efficiency results in a detectable modulation of emission intensity or quenching.

In another strategy, FRET efficiency may be modulated by varying the number of fluorescent labels or quenchers attached to each incoming nucleotide. In this strategy, differing numbers of the same fluorescent label or quencher are attached to each nucleotide. For example, one fluorescent label may be attached to A, two to T, three to G, and four to C. Increasing the number of acceptors relative to the nanoparticle donors increases FRET efficiency and quantum yield, such that base discrimination may be based on the intensity of light emission from the acceptor(s) or the reduction of light emission from the nanoparticle donor(s).

In another embodiment, the nucleotide comprises a releasable label that can be removed via suitable means prior to incorporation of the next nucleotide by the polymerase into the newly synthesized strand. The use of releasably labeled nucleotides wherein the label can be cleaved and removed via suitable means have been described, for example, in U.S. Pub. Nos. US2005/0244827 and US2004/0244827, as well as U.S. Pat. Nos. 7,345,159; 6,664,079; 7,345,159; and 7,223,568.

Preferably, the label of the polymerase and the label of the nucleotide will be selected and/or designed to ensure not that the presence of such labels does not unduly hinder the progress of the polymerization reaction as determined by speed, error rate, fidelity, processivity and average read length of the newly synthesized strand.

In a preferred embodiment, a suitable primer is included in the nucleic acid polymerase reaction. The primer length is typically determined by the specificity desired for binding the complementary template as well as the stringency of the annealing and reannealing conditions employed. The primer can comprise ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, and modified phosphate-sugar backbone nucleotides, and any analogs or variants of the foregoing compounds. The primer can be synthetic, or produced naturally by primases, RNA polymerases, or other oligonucleotide synthesizing enzymes. The primer may be any suitable length including at least 5 nucleotides, 5 to 10, 15, 20, 25, 50, 75, 100 nucleotides or longer in length. In a preferred embodiment, the polymerase extends the primer by a plurality of nucleotides. Optionally, the primer is extended at least 50, 100, 250, 500, 1000, or at least 2000 nucleotide monomers.

Alternatively, the initiation site for sequencing can be created through any suitable means without requiring use of a primer. For example, the polymer to be sequenced may comprise, or be associated with, a polymerase priming site capable of extension via polymerization of monomers by the polymerase. The priming site may be generated, for example, by treatment of the polymer so as to produce nicks or cleavage sites. Yet another option is for the target polymer to undergo “hairpin” formation, either through annealing to a self-complementary region within the target sequence itself or through ligation to a self-complementary sequence, resulting in a structure that undergoes self-priming under suitable conditions.

Typically, the sequencing reaction is initiated by the addition of a suitable polymerase and labeled nucleotides. Suitable temperatures and the addition of other components such as divalent metal ions can be determined and optimized based on the particular nucleotide polymerase and the target nucleic acid sequences. Illumination of the reaction site permits observation of the detectable signals, e.g., FRET signals, which indicate the nucleotide incorporation event.

The signals emitted by various components of the polymerase reaction mixture as the polymerase incorporates nucleotide(s) into an elongating strand in a template-directed fashion can be detected by means of any suitable system capable of detecting and/or monitoring such signals. Typically, the detection system will achieve these functions by first generating and transmitting an incident wavelength to the polynucleotides isolated within nanostructures, and then collecting and analyzing the emissions from the reactants.

The identities of the incorporated nucleotides may be determined rapidly, for example in real-time or near real-time, as extension of the primer strand occurs, through FRET interactions between the semiconductor nanoparticle, nanocrystal or quantum dot (i.e., the donor) attached to the primer and a label (i.e., an acceptor) attached to the incoming nucleotides as they are incorporated into the complementary strand. The nucleotides used for extension of the primer in the present disclosure are labeled with either a fluorescent label, a quencher, or some combination thereof. In some embodiments, the label is attached to a phosphate, for example the β-phosphate, the γ-phosphate, or the terminal phosphate of the nucleotide, such that the label is separated from the nucleotide upon incorporation into primer strand by the nucleic acid polymerase. In other embodiments, the label is attached to the α-phosphate, the nitrogenous base, or the sugar of the nucleotide and used in combination with a quencher. As discussed below, a number of labeling and detection strategies are available to determine the identity of the nitrogenous base of the incoming nucleotides.

All of these strategies rely on FRET between the semiconductor donor attached to the primer and the fluorescent label and/or quencher acceptor attached to the incoming nucleotide. In the present disclosure, the quantum dot donor is excited by illumination with light of an appropriate excitation wavelength, as required by the excitation spectrum of the quantum dot. Given the exceptional photostability of quantum dots, continuous excitation without photobleaching is possible. As the nucleotide polymerase incorporates incoming nucleotides complementary to the target nucleic acid molecule into the primer strand, the label attached to the nucleotide is brought into close proximity with the quantum dot. When the distance between the quantum dot and label decreases to approximately 1.0 to 1.5×R₀ or less, FRET efficiency increases sufficiently to trigger detectable FRET between the quantum dot and label, either through the emission of light from the label or quenching of the quantum dot's light emission.

Detection of the FRET signal and spectral resolution permitting discrimination between the various nucleotide signals can be achieved using any suitable method including spectral wavelength analysis, correlation/anti-correlation analysis, fluorescent lifetime measurement, and fluorophore identification. Suitable techniques for detecting the emissions include confocal laser scanning microscopy, Total Internal Reflection Fluorescence (TIRF) and other forms of fluorescence microscopy.

In certain embodiments, the label is attached to a phosphate that is cleaved by the polymerase from the nucleotide upon incorporation into the complementary sequence, for example the β-phosphate, the γ-phosphate, or the terminal phosphate of the incoming nucleotide. By cleaving the phosphate and releasing the label upon incorporation of the incoming nucleotide, the FRET signal between the quantum dot and the label ceases after the nucleotide is incorporated and the label diffuses away. Thus, in these embodiments, a FRET signal is generated as each incoming nucleotide hybridizes to a complementary nucleic acid in the target nucleic acid molecule, and upon incorporation of the nucleotide into the elongating primer strand, the label is released and the FRET signal ends. By releasing the label upon incorporation, successive extensions can each be detected without interference from nucleotides previously incorporated into the complementary strand.

Typically, the Förster distance (R₀) depends in part on the specific combination of FRET donor and acceptor used. In some embodiments up to about 10, 20, 30, 40, 50, 75 or 100 nucleotides may be sequenced using the methods and compositions disclosed herein.

A number of labeling and detection strategies are available for base discrimination using the FRET technique. For example, different fluorescent labels may be used for each nucleotide in the reaction mixture (for each type of nucleotide present in the extension reaction), with discrimination between the different labels based on the wavelength and/or the intensity of the light emitted from the fluorescent label.

Any suitable materials may be used for the solid support. Exemplary materials include, but are not limited to, glass, plastic, glass with surface modifications, silicon, metals, semiconductors, high refractive index dielectrics, nylon, nitrocellulose, PVDF, crystals, gels, and polymers. The solid support can be in any format including plate, microarray, sheet, filter, and beads. Techniques for binding the target nucleic acid molecule and/or primer to the substrate are determined by the materials employed. For example, binding partners such as streptavidin can be employed with biotinylated template or primer. Reversible or irreversible binding between the support and either the nanoparticle-labeled primer or the target nucleic acid sequence can be achieved with the components of any suitable covalent or non-covalent binding pair. Other such suitable immobilization approaches for immobilizing can include an antibody (or antibody fragment)-antigen binding pair and photoactivated coupling molecules. Generally, suitable immobilization can be applied to the support by conventional chemical and photolithographic techniques which are well known in the art and include standard chemical surface modifications of the solid support, and support incubation using differential temperatures and media.

In some embodiments, individual polymeric molecules are first isolated using a nanofluidic device comprising a nanochannel array, wherein the entire sample population is elongated and displayed in a spatially addressable format. As disclosed herein, the use of nanofluidic devices to isolate and sequence a target polymer of interest, in combination with signal analysis provides significant advantages. For example, the use of nanofluidic devices for separation and isolation of test polymeric molecules bypasses the requirement for immobilization or attachment of sequencing components to a substrate and also enables the sequencing of intact chromosomes, thereby exponentially increasing the amount of sequencing information obtained from a single reaction and also enabling analysis of such “macro” structural features as methylation, inversions, indels and tandem repeats. In some embodiments, nanofluidic devices that permit the simultaneous observation of a high number of macromolecules in a multitude of channels can be employed. Such devices increase the amount of sequence information obtainable from a single experiment and decrease the cost of sequencing of an entire genome. See, for example, U.S. Pub. No. 2004/0197843 and 2004/0166025; U.S. Pat. Nos. 6,696,022; 6,762,059 and 6,927,065. Furthermore, by using nanoparticles or analogs thereof operably linked to polymerase activity, polymer sequence data can be generated as labeled monomers are incorporated into a newly synthesized polymer strand by a polymerase, thus enabling the sequencing of polymers in real time. Moreover, the nanofluidic-based sequencing methods disclosed herein can be used to rapidly obtain both “raw” sequence at the single nucleic acid molecule level as well as validation of incoming sequence information via simultaneous priming at multiple points along the template strand.

Also disclosed herein are methods for sequencing polymeric molecules isolated through nanofluidic manipulation. Isolation of the test molecules to be sequenced may be achieved using any suitable nanofluidic device that comprises nanostructures or nanofluidic constrictions of a size suited to achieve isolation and separation of the test polymer from other sample components in a manner that will support direct sequencing of the polymer in situ. In some embodiments a polymeric molecule, such as the DNA of an entire chromosome, can be isolated from a sample mixture using a nanofluidic device that is capable of receiving a sample comprising mixed population of polymers and elongating and displaying them in an ordered format without the need for prior treatment or chemical attachment to a support. In some embodiments, the nanofluidic device comprises at least one nanostructure, typically a nanochannel, which is designed to admit only a single polymeric molecule and elongate it as it flows through the nanostructure. Suitable nanofluidic devices that may be used to practice the disclosed methods, systems and/or compositions are described, for example, in U.S. Pat. No. 6,635,163; U.S. Pat. No. 7,217,562, U.S. Pub. No. 2004/0197843 and U.S. Pub. No. 2007/0020772. In some embodiments, the nanostructures of the nanofluidic device can optionally satisfy any one, some or all of three requirements: (1) they can have a sufficiently small dimension to elongate and isolate macromolecules; (2) they can be sufficient length to permit instantaneous observation of the entire elongated macromolecule; and (3) the nanochannels or other nanostructures can be sufficiently numerous to permit simultaneous and parallel observation of a large population of macromolecules. In one embodiment, the radius of the component nanostructures of the nanofluidic device will be roughly equal to or less than the persistence length of the target DNA.

In one embodiment, the nanofluidic device comprises an array of nanochannels. Introduction of a sample comprising a mixed population of polymeric molecules into the nanofluidic device results in the isolation and elongation of a single polymeric molecule within each nanochannel, so that the entire population of polymeric molecules displayed in an elongated and spatially addressable format. After each polymer enters and flows through its respective nanochannel, it is contacted with one or more components of a polymerase reaction mixture, so that separate sequencing reactions occur within each nanochannel. The progress of the sequencing reaction is monitored using suitable detection methods. The ordered and spatially addressable arrangement of the population allows signals to be detected and monitored along the length of each polymeric molecule, and also permits discrimination of signals generated by separate priming events, thus permitting simultaneous detection and analysis of multiple priming events at multiple points in the array. The emission data is gathered and analyzed to determine the time-sequence of incorporation events for each individual DNA in the nanochannel array.

As disclosed herein, the use of nanofluidic devices to isolate and sequence a target polymer of interest, in combination with signal analysis provides significant advantages. For example, the use of nanofluidic devices for separation and isolation of test polymeric molecules bypasses the requirement for immobilization or attachment of sequencing components to a substrate and also enables the sequencing of intact chromosomes, thereby exponentially increasing the amount of sequencing information obtained from a single reaction and also enabling analysis of such “macro” structural features as methylation, inversions, indels and tandem repeats. In some embodiments, nanofluidic devices that permit the simultaneous observation of a high number of macromolecules in a multitude of channels can be employed, thereby increasing the amount of sequence information obtainable from a single experiment and decreasing the cost of sequencing of an entire genome. See, for example, U.S. Pub. Nos. 2004/0197843 and 2004/0166025; U.S. Pat. Nos. 6,696,022; 6,762,059 and 6,927,065. Furthermore, by using semiconductor nanocrystals operably linked to polymerase activity, polymer sequence data can be generated as labeled monomers are incorporated into a newly synthesized polymer strand by a polymerase, thus enabling the sequencing of polymers in real time. Moreover, the nanofluidic-based sequencing methods disclosed herein can be used to rapidly obtain both “raw” sequence at the single nucleic acid molecule level as well as validation of incoming sequence information via simultaneous priming at multiple points along the template strand.

Also suitable for use according to the present disclosure are modified nanofluidic devices comprising microfluidic and nanofluidic areas separated by a gradient interface that reduces the local entropic barrier to nanochannel entry and thereby decreases clogging of the device at the microfluidic-nanofluidic interface. See, for example, U.S. Pat. No. 7,217,562 and U.S. Pub. No. 2007/0020772.

In some embodiments, the nanofluidic device supports analysis of entire, intact chromosomes without need for fragmentation or immobilization to a substrate of the nucleic acid or polymer being sequenced.

In some embodiments, the nanofluidic device comprises a plurality of nanochannels, typically more than 5, 10, 100, 1000, 10,000 and 100,000 nanochannels.

Suitable nanofluidic devices may be fabricated from any suitable substrate (including, but not limited to silicon, carbon, glass, polymers, metals, boron nitrides and synthetic vesicles) using any suitable method, including, but not limited to lithography, photolithography, diffraction gradient lithography, nanoimprint lithography, interference lithography, self-assembled copolymer pattern transfer, spin coating, electron beam lithography, focused ion beam milling, plasma-enhanced chemical vapor deposition, electron beam evaporation, sputter deposition, bulk or surface micromachining, replication techniques such as embossing, printing, casting and injection molding), etching (including but not limited to nuclear track or chemical etching, reactive ion-etching and wet-etching), and combinations thereof.

Suitable nanostructures for inclusion in the nanofluidic device include, but are not limited to, single cylindrical channels, nanoslits, nanochannels, nanopores and nanopillars. In a preferred embodiment, the nanostructure comprises one or more nanochannels capable of transporting a macromolecule across their entire length in elongated form. Typically, the nanochannels are in array format. Optionally, the nanochannels may be substantially enclosed by surmounting them with a sealing material using suitable methods. See, for example, U.S. Pub. No. 2004/0197843. In some embodiments, the dimension of the nanochannels will be equal to or lesser than the persistence length of the test polymer to be isolated. In some embodiments, the nanochannels will have a trench width equal to or less than about 150 nanometers, and a trench depth equal to or less than about 200 nanometers. Optionally, the nanofluidic device may further comprise a sample reservoir capable of releasing a fluid, and a waste reservoir capable of receiving a fluid, wherein both reservoirs are in fluid communication with the nanofluidic area. The nanofluidic device may optionally comprise a microfluidic area located adjacent to the nanofluidic area, and a gradient interface between the microfluidic and nanofluidic area that reduces the local entropic barrier to nanochannel entry. See, for example, U.S. Pat. No. 7,217,562.

The detection system typically comprises at least two elements, namely an excitation source and a detector. The excitation source generates and transmits incident radiation used to excite the reactants contained in the array. Depending on the intended application, the source of the incident light can be a laser, laser diode, a light-emitting diode (LED), a ultra-violet light bulb, and/or a white light source. Where desired, more than one source can be employed simultaneously. The use of multiple sources is particularly desirable in applications that employ multiple different reagent compounds having differing excitation spectra, consequently allowing detection of more than one fluorescent signal to track the interactions of more than one or one type of molecules simultaneously.

Any suitable detection strategies can be employed to determine the identity of the nitrogenous base of the incoming nucleotides, depending on the nature of the labeling strategy that is employed. Exemplary labeling and detection strategies include but are not limited to those disclosed in U.S. Pat. Nos. 6,423,551 and 6,864,626; U.S. Pub. Nos. 2005/0003464, 2006/0176479, 2006/0177495, 2007/0109536, 2007/0111350, 2007/0116868, 2007/0250274 and 2008/08825. Detection of emissions during the polymerization reaction permits the discrimination of independent interactions between uniquely labeled moieties, reactants or subunits. On exposure to suitable chemical, electrical, electromagnetic energy (potentially any light source, typically a laser) or upon resonance as in FRET, the label linked to the nucleotide undergoes a transition to an ‘excited state’ whereby it emits photons over a spectral range characterized by the identity of the emitting moiety. The donor moiety must be sufficiently excited in order for FRET to occur.

Emissions may be detected using any suitable device. A wide variety of detectors are available in the art. Representative detectors include but are not limited to optical readers, high-efficiency photon detection systems, photodiodes (e.g. avalanche photo diodes (APD); APD arrays, etc.), cameras, charge couple devices (CCD), electron-multiplying charge-coupled device (EMCCD), intensified charge coupled device (ICCD), photomultiplier tubes (PMT), a muti-anode PMT, and a confocal microscope equipped with any of the foregoing detectors. Where desired, the subject arrays contain various alignment aides or keys to facilitate a proper spatial placement of each spatially addressable array location and the excitation sources, the photon detectors, or the optical transmission element as described below.

Typically, characteristic signals from different independently labeled, nucleotides are simultaneously detected and resolved using a suitable detection method capable of discriminating between the respective labels. Typically, the characteristic signals from each nucleotide are distinguished by resolving the characteristic spectral properties of the different labels. See, for example, Lakowitz, J. R., 2006, Principles of Fluorescence Spectroscopy, Third Edition. Spectral detection may also optionally be combined and/or replaced by other detection methods capable of discriminating between chemically similar or different labels in parallel, including, but not limited to, polarization, lifetime, Raman, intensity, ratiometric, time-resolved anisotropy, fluorescence recovery after photobleaching (FRAP) and parallel multi-color imaging. See, for example, Lakowitz, supra. In the latter technique, use of an image splitter (such as, for example, a dichroic mirror, filter, grating, prism, etc.) to separate the spectral components characteristic of each label is preferred to allow the same detector, typically a CCD, to collect the images in parallel. Optionally, multiple cameras or detectors may be used to view the sample through optical elements (such as, for example, dichroic mirrors, filters, gratings, prisms, etc.) of different wavelength specificity. Other suitable methods to distinguish emission events include, but are not limited to, correlation/anti-correlation analysis, fluorescent lifetime measurements, anisotropy, time-resolved methods and polarization detection. Suitable imaging methodologies that may be implemented for detection of emissions include, but are not limited to, confocal laser scanning microscopy, Total Internal Reflection (TIR), Total Internal Reflection Fluorescence (TIRF), near-field scanning microscopy, far-field confocal microscopy, wide-field epi-illumination, light scattering, dark field microscopy, photoconversion, wide field fluorescence, single and/or multi-photon excitation, spectral wavelength discrimination, evanescent wave illumination, scanning two-photon, scanning wide field two-photon, Nipkow spinning disc, multi-foci multi-photon, and/or other forms of microscopy.

The detection system may optionally include one or more optical transmission elements that serve to collect and/or direct the incident wavelength to the reactant array; to transmit and/or direct the signals emitted from the reactants to the photon detector; and/or to select and modify the optical properties of the incident wavelengths or the emitted wavelengths from the reactants. Illustrative examples of suitable optical transmission elements and optical detection systems include but are not limited to diffraction gratings, arrayed wave guide gratings (AWG), optic fibers, optical switches, mirrors, lenses (including microlens and nanolens), collimators. Other examples include optical attenuators, polarization filters (e.g., dichroic filters), wavelength filters (low-pass, band-pass, or high-pass), wave-plates, and delay lines.

Typically, the detection system comprises optical transmission elements suitable for channeling light from one location to another in either an altered or unaltered state. Non-limiting examples of such optical transmission devices include optical fibers, diffraction gratings, arrayed waveguide gratings (AWG), optical switches, mirrors, (including dichroic mirrors), lenses (including microlens and nanolens), collimators, filters, prisms, and any other devices that guide the transmission of light through proper refractive indices and geometries.

In one embodiment, the detection system comprises an optical train that directs signals from an organized array onto different locations of an array-based detector to simultaneously detect multiple different optical signals from each of multiple different locations. In particular, the optical trains typically include optical gratings and/or wedge prisms to simultaneously direct and separate signals having differing spectral characteristics from each spatially addressable location in an array to different locations on an array-based detector, e.g., a CCD. By separately directing signals from each array location to different locations on a detector, and additionally separating the component signals from each array location, one can simultaneously monitor multiple signals from each array location.

In a preferred embodiment, detection is performed using multifluorescence imaging wherein each of the different types of nucleotide is operably linked to a label with different spectral properties from the rest, thereby permitting the simultaneous detection of incorporation of all different nucleotide types. For example, each of the different types of nucleotide may be operably linked to a FRET acceptor fluorophore, wherein each fluorophore has been selected such that the overlapping of the absorption and emission spectra between the different fluorophores, as well as the overlapping between the absorption and emission maxima of the different fluorophores, is minimized. Detection of different nucleotide label is performed by observing two or more targets at the same time, wherein the emissions from each label are separated in the detection path. Such separation is typically accomplished through use of suitable filters, including but not limited to band pass filters, image splitting prisms, band cutoff filters, wavelength dispersion prisms and dichroic mirrors, that can selectively detect specific emission wavelengths. Such filters may optionally be used in combination with suitable diffraction gratings.

Alternatively, multifluorescence studies involving differently labeled nucleotide types may be performed by observing each label separately, requiring section of special filter combinations for each excitation line and each emission band. In one embodiment, the detection system utilizes tunable excitation and/or tunable emission fluorescence imaging. For tunable excitation, light from a light source passes through a tuning section and condenser prior to irradiating the sample. For tunable emissions, emissions from the sample are imaged onto a detector after passing through imaging optics and a tuning section. The user may control the tuning sections to optimize performance of the system.

A number of labeling and detection strategies are available for base discrimination using the FRET technique. For example, different fluorescent labels may be used for each type of nucleotide present in the extension reaction with discrimination between the different labels based on the wavelength and/or the intensity of the light emitted from the fluorescent label.

A second strategy involves the use of fluorescent labels and quenchers. In this strategy, certain nucleotides in the reaction mixture are labeled with a fluorescent label, while the remaining nucleotides are labeled with one or more quenchers. Alternatively, each of the nucleotides in the reaction mixture is labeled with one or more quenchers. Discrimination of the nucleotide bases is based on the wavelength and/or intensity of light emitted from the FRET acceptor, as well as the intensity of light emitted from the FRET donor. If no signal is detected from the FRET acceptor, a corresponding reduction in light emission from the FRET donor indicates incorporation of a nucleotide labeled with a quencher. The degree of intensity reduction may be used to distinguish between different quenchers.

A third strategy involves modulating FRET efficiency by varying the distance between the nanoparticle donor and the fluorescent label or quencher acceptor. In this strategy, the same type of fluorescent label or quencher may be used, however, the distance between the nanoparticle and the label is varied for each nucleotide to be identified, causing a modulation of FRET efficiency. The distance may be varied through the structure of the nucleotide itself, the position of the label or quencher on the nucleotide, or the use of spacers or linkers during attachment of the fluorescent label or quencher to the nucleotide. Modulation of FRET efficiency results in a detectable modulation of emission intensity or quenching.

In another strategy, FRET efficiency may be modulated by varying the number of labels or quenchers attached to each incoming nucleotide. In this strategy, differing numbers of the same label or quencher are attached to each nucleotide. For example, one label may be attached to A, two to T, three to G, and four to C. Increasing the number of acceptors relative to the nanoparticle donors increases FRET efficiency and quantum yield, such that base discrimination may be based on the intensity of light emission from the acceptor(s) or the reduction of light emission from the nanoparticle donor(s).

Typically, the signal from the detector is converted into a digital signal with an A-D converter and an image of the sample is reconstructed on a monitor. The user can optionally select a composite image that combines the images derived at a number of different wavelengths into a single image. The user can also specify that an artificial color system is to be used in which particular probes are artificially associated with specific colors. In an alternate artificial color system the user can designate specific colors for specific emission intensities.

Any combination of the above described labeling and detection strategies may be employed together in the same sequencing reaction. Depending on the number of distinguishable labels and quenchers used in any of the above strategies, the identities of one, two, or four nucleotides may be determined in a single sequencing reaction. Multiple sequencing reactions may then be run, rotating the identities of the nucleotides determined in each reaction, to determine the identities of the remaining nucleotides. In some embodiments, these reactions may be run at the same time, in parallel, to allow for complete sequencing in a reduced amount of time.

The identities of the incorporated nucleotides may be determined rapidly, for example in real time or near real time, as extension of the primer strand occurs, through FRET interactions between a nanoparticle attached to the polymerase, typically at or near the reaction site and a FRET acceptor moiety attached to the incoming nucleotides as they are incorporated into the complementary strand.

Typically, the raw data generated by the detector represents between multiple time-dependent fluorescence data stream comprising wavelength and intensity information. Once the emissions are detected and gathered, the data may be analyzed using suitable methods to correlate the particular spectral characteristics of the emissions with the identity of the incorporated base. In some embodiments, such analysis is performed by means of a suitable information processing and control system. Preferably, the information processing and control system comprises a computer or microprocessor attached to or incorporating a data storage unit containing data collected from the detection system. The information processing and control system may maintain a database associating specific spectral emission characteristics with specific nucleotides. The information processing and control system may record the emissions detected by the detector and may correlate those emissions with incorporation of a particular nucleotide. The information processing and control system may also maintain a record of nucleotide incorporations that indicates the sequence of the template molecule. The information processing and control system may also perform standard procedures known in the art, such as subtraction of background signals.

An exemplary information processing and control system may incorporate a computer comprising a bus for communicating information and a processor for processing information. In one embodiment, the processor is selected from the Pentium®, Celeron®, Itanium®, or a Pentium Xeon® family of processors (Intel Corp., Santa Clara, Calif.). Alternatively, other processors may be used. The computer may further comprise a random access memory (RAM) or other dynamic storage device, a read only memory (ROM) and/or other static storage and a data storage device such as a magnetic disk or optical disc and its corresponding drive. The information processing and control system may also comprise other peripheral devices known in the art, such a display device (e.g., cathode ray tube or Liquid Crystal Display), an alphanumeric input device (e.g., keyboard), a cursor control device (e.g., mouse, trackball, or cursor direction keys) and a communication device (e.g., modem, network interface card, or interface device used for coupling to Ethernet, token ring, or other types of networks).

In particular embodiments, the detection system may also be coupled to the bus. Data from the detection unit may be processed by the processor and the data stored in the main memory. Data on emission profiles for standard nucleotides may also be stored in main memory or in ROM. The processor may compare the emission spectra from nucleotide in the polymerase reaction to identify the type of nucleotide precursor incorporated into the newly synthesized strand. The processor may analyze the data from the detection system to determine the sequence of the template nucleic acid.

It is appreciated that a differently equipped information processing and control system than the example described above may be used for certain implementations. Therefore, the configuration of the system may vary in different embodiments. It should also be noted that, while the processes described herein may be performed under the control of a programmed processor, in alternative embodiments, the processes may be fully or partially implemented by any programmable or hardcoded logic, such as Field Programmable Gate Arrays (FPGAs), TTL logic, or Application Specific Integrated Circuits (ASICs), for example. Additionally, the method may be performed by any combination of programmed general purpose computer components and/or custom hardware components.

Following the data gathering operation, the data will typically be reported to a data analysis operation. To facilitate the analysis operation, the data obtained by the detection system will typically be analyzed using a digital computer. Typically, the computer will be appropriately programmed for receipt and storage of the data from the detection system, as well as for analysis and reporting of the data gathered.

Any suitable base-calling algorithms may be employed. See, for example, US. Provisional App. No. 61/037,285. In certain embodiments, custom designed software packages may be used to analyze the data obtained from the detection system. In alternative embodiments, data analysis may be performed, using an information processing and control system and publicly available software packages. Non-limiting examples of available software for DNA sequence analysis include the PRISM™ DNA Sequencing Analysis Software (Applied Biosystems, Foster City, Calif.), the Sequencher™ package (Gene Codes, Ann Arbor, Mich.), and a variety of software packages available through the National Biotechnology Information Facility at website www.nbif.org/links/1.4.1.php. Data collection allows data to be assembled from partial information to obtain sequence information from multiple polymerase molecules in order to determine the overall sequence of the template or target molecule.

Additionally, in certain instances it is useful to perform reactions with reference controls, similar to microarray assays. Comparison of signal(s) between the reference sequence and the test sample are used to identify differences and similarities in sequences or sequence composition. Such reactions can be used for fast screening of DNA polymers to determine degrees of homology between the polymers, to determine polymorphisms in DNA polymers, or to identity pathogens.

In some embodiments, the method further comprises sequencing one or more additional nucleic acid molecules, for example a second nucleic acid, in parallel with sequencing the first nucleic acid. In other embodiments, the rate of nucleotide sequencing determination (based on a single read of a nucleic acid template) is equal to or greater than 10 nucleotides per second, typically equal to or greater than 100 nucleotides per second.

Typically, the sequencing error rate will be equal to or less than 1 in 100,000 bases. In some embodiments, the error rate of nucleotide sequence determination is equal to or less than 1 in 10 bases, 1 in 20 bases, 3 in 100 bases, 1 in 100 bases, 1 in 1000 bases, and 1 in 10,000 bases. In another preferred embodiment, the test DNA will comprise a complete and intact chromosome. Optionally, the methods disclosed herein may be performed in a multiplex fashion (including in array format), such that additional nucleic acid molecules are sequenced in parallel with a first nucleic acid molecule.

All of the compositions, systems and methods disclosed and/or claimed herein can be made and executed without undue experimentation in light of the present disclosure. While the compositions, systems and methods of this disclosure have been described in terms of preferred embodiments, these embodiments are in no way intended to limit the scope of the appended claims, and it will be apparent to those of skill in the art that variations may be applied to the compositions and/or methods and in the steps or in the sequence of steps of the methods described herein without departing from the concept, spirit and scope of this disclosure. More specifically, it will be apparent that certain agents, compositions, systems or methods which are chemically, physiologically or functionally related may be substituted for the agents, compositions, systems or methods described herein while the same or similar results would be achieved. All such similar substitutions and modifications apparent to those skilled in the art are deemed to be within the spirit, scope and concept of this disclosure and the appended claims.

### Example 1

### Sequencing of a Single Target DNA in Real Time

A. Isolation of Test DNA within a Nanochannel

Intact chromosomal DNA is extracted from a suitable tissue source using standard methods, and diluted to an appropriate concentration (0.1-0.5 microgram/mL) in 0.5×TBE buffer. The test DNA is conjugated to a self-complementary sequence capable of undergoing “hairpin” formation, and ligated products are purified using standard techniques. The purified ligated product is placed in a plastic delivery tube placed in fluid communication with a prewetted nanofluidic device comprising a sample reservoir feeding a nanofluidic area. The nanofluidic area comprises nanochannels as disclosed in U.S. Pat. No. 7,217,562 and U.S. Pub. Nos. 2007/0020772 and 2004/0197843. The DNA is introduced into the array by electric field (at 1-50 V/cm). After a suitable interval, each nanochannel contains a single test DNA, such that the entire sample population of DNA molecules is elongated and displayed in any array format. The displayed population is then subjected in situ to the sequencing process described below.

B. Conjugation of DNA Polymerase to a Semiconductor Nanocrystal

A DNA construct encoding DNA polymerase was constructed and used to express and purify DNA polymerase in vitro using standard techniques. The purified DNA polymerase preparation was then conjugated with a semiconductor nanocrystal. In some experiments, the purified polymerase was conjugated to the nanocrystal using affinity coupling, via coincubation of the polymerase with a suspension of nanocrystals previously functionalized via attachment of dipeptide residues to the surface of the nanocrystal. In other experiments, conjugation of the nanocrystal with the polymerase was achieved through use of the linker succinimidyl-4-(N-maleimidomethyl)cyclohexane-1-carboxylate, also known as SMCC. This linker contains a maleimide reactive group that reacts specifically with a suitable thiol group on the purified protein, while the opposite terminus contains an amine-reactive group that reacts with amines on the nanocrystal. Following the conjugation reaction, conjugates were separated from unconjugated components using size exclusion chromatography, following which a purified suspension of Klenow-Nanocrystal conjugates were eluted from the column.

C. Characterization of Polymerase-Nanocrystal Conjugates

In preliminary experiments, the purified Klenow-Nanocrystal conjugates were analyzed to assess enzymatic activity and excitation behavior. First, binding assays were performed by co-incubating a double-stranded fluorescently labeled DNA molecule with the purified Klenow:Nanocrystal conjugates. The binding process was experimentally following by monitoring various fluorescence characteristics of the DNA label, including fluorescence polarization and fluorescence intensity.

Separately, primer extension reactions were performed using the Klenow-Nanocrystal conjugates. Briefly, a 3′ dye-labeled primer was hybridized to a DNA target, and the resultant hybrid was co-incubated with deoxynucleotide triphosphates and the purified Klenow-Nanocrystal conjugates. The progress of primer extension was monitored by detecting and analyzing changes in fluorescence polarization and fluorescence intensity of the dye label.

D. Initiation of Sequencing Reaction

Dye labels were conjugated to the terminal phosphate of deoxyribonucleotide polyphosphates using a linker and/or spacer using standard techniques. The nanofluidic device comprising nanochannels containing isolated test DNA molecules was flushed with a reaction mixture comprising labeled nucleotides and purified Klenow-Nanocrystal conjugates, prepared according to the procedures described herein. Simultaneously, the nanofluidic device was irradiated at the excitation wavelength for the nanocrystal. Within the nanochannels, the conjugated Klenow polymerase begins to extend the 3′ end of the hairpin structure via successive addition of labeled dN4P residues to form a newly synthesized strand that is complementary to the test DNA of interest.

As the labeled dN4P enters the nucleotide binding site, it is brought into proximity with the nanocrystal on the polymerase, resulting in Forster resonance energy transfer (FRET) from the nanocrystal to the FRET acceptor on the incoming dN4P. Following ligation of the incoming nucleotide tetraphosphate to the 3′ end of the elongated strand, the labeled phosphate is cleaved off and the polymerase-nanocrystal conjugate translocates to a new position on the template strand. Thus, a FRET signal is generated as each incoming nucleotide hybridizes to a complementary nucleotide in the target nucleic acid molecule, and upon incorporation of the nucleotide into the elongating primer strand, the label is released and the FRET signal ends.

E. Detection and Analysis of Resonance Energy Transfer (Fret) Between the Labeled Polymerase and Labeled Nucleotide Monomer

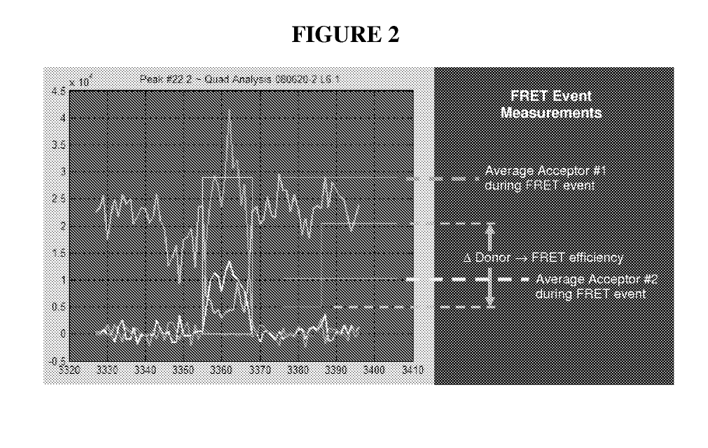

Donor signals required for FRET are generated by illumination with appropriate excitation source (e.g. laser) of a microfluidic chamber containing the reaction components. The identity of incorporated nucleotides is revealed by the simultaneous real-time detection of FRET signals that arise from nucleotide (or polymer) subunits independently labeled with spectrally distinct luminophores. These events are detected using techniques as disclosed in U.S. Pub. No. 2007/0250274. Total Internal Reflection (TIR) microscopy is used to visualize emissions according to standard methods (Axelrod, 1985). Using an image splitter (e.g. dichroic mirrors, filters), the spectral components characteristic of the luminophores are separated and collected or imaged on a CCD. Time resolved data streams are collected and stored and subsequently processed to remove background “noise”. See, for example, FIG. 2. Using suitable base-calling algorithms, the data is analyzed to correlate various FRET donor:acceptor pairs, and thereby reveal the identity of time-resolved independent dNTP incorporation events into the elongating strand.

1. A method for determining in real time a nucleotide sequence of a nucleic acid molecule, comprising the steps of:
(a) isolating a single nucleic acid molecule in a nanochannel of a nanofluidic device;
(b) conducting in the nanochannel a polymerase reaction in the presence of at least one detectably-labeled nucleotide or nucleotide analog, which reaction results in the production of a detectable signal indicating incorporation of the at least one detectably-labeled nucleotide or nucleotide analog into a growing nucleotide strand by the polymerase;
(c) detecting a time sequence of nucleotide or nucleotide analog incorporations; and
(d) determining the identity of one or more nucleotides or nucleotide analogs incorporated during the polymerase reaction, thereby determining some or all of the nucleotide sequence of the nucleic acid molecule.

2. The method of claim 1, wherein the detectable label is a detectable label linked to a terminal phosphate in the polyphosphate chain of the detectably-labeled nucleotide, which reaction results in the production of a labeled polyphosphate that is released from the detectable terminal-phosphate labeled nucleotide.

3. The method of claim 1, wherein the nanofluidic device further comprises a nanochannel array.

4. The method of claim 1, wherein the nanofluidic device further comprises a nanochannel array comprising 100 or more nanochannels.

5.-11. (canceled)

12. The method of claim 1, wherein the nanofluidic device further comprises one or more nanochannels capable of transporting a macromolecule across their length.

13. The method of claim 12, wherein the macromolecule is transported across the one or more nanochannels in an elongated form.

14.-16. (canceled)

17. The method of claim 1, wherein the detectable label of the detectably-labeled nucleotide is a Forster resonance energy transfer (FRET) acceptor.

18. The method of claim 1, wherein the detectable signal is produced as a result of Forster resonance energy transfer (FRET) from a FRET donor to the FRET acceptor.

19.-22. (canceled)

23. The method of claim 1, wherein the nucleic acid polymerase of the nucleic acid polymerase reaction is operably linked to a Forster resonance energy transfer (FRET) donor.

24. The method of claim 23, wherein the FRET donor is a nanocrystal.

25.-57. (canceled)
